(19) United States
(12) Patent Application Publication
(10) Pub. No.: US 2010/0099961 A1
(43) Pub. Date: Apr. 22, 2010

(54) METHOD FOR DETERMINING MICROVASCULAR LESIONS

(75) Inventors: Thomas Hubner, Jena (DE); Michael Alt, Kirchheim (DE)

Correspondence Address: MAIER & MAIER, PLLC, 1000 DUKE STREET, ALEXANDRIA, VA, 22314, US

(73) Assignee: Enverdis GmbH, Jena (DE)

(21) Appl. No.: 12/532,312

(22) PCT Filed: Mar. 20, 2008
(86) PCT No.: PCT/EP2008/053412

§ 371 Date: Sep. 21, 2009

(30) Foreign Application Priority Data
Mar. 23, 2007 (EP) 07104768.2

(51) Int. Cl. A61B 5/1455; A61B 5/02

(52) U.S. Cl. 600/322; 600/504

(57) ABSTRACT

The invention relates to a method for determining microvascular diseases wherein, first, a volume pulse development of a first blood constituent of the blood in a blood path is determined, and a volume pulse development of a second blood constituent in a blood path, being different from the first blood constituent, is determined. Thereafter, comparable reference points in the volume pulse development of the first blood constituent and in the volume pulse development of the second blood constituent are determined. Then, there is determined a temporal difference (Δt) of the determined reference points in the volume pulse developments of the first blood constituent and the second blood constituent.

### RELATED APPLICATIONS

The present invention claims priority under 35 U.S.C. §119 to European Patent Application No. 07104768.2, filed on Mar. 23, 2007, the disclosure of which is incorporated by reference herein in its entirety.

### BACKGROUND

The invention relates to a method for the non-invasive determining of microvascular diseases of the arterioles and capillaries.

Such lesions can be caused e.g. by arteriosclerosis or a diabetes-conditioned microangiopathy.

Arteriosclerosis denotes a chronic disease of the arteries wherein fat becomes deposited on the intima (intima=innermost layer of the vessel wall), the vessel walls will then calcify and thereby lose their elasticity (sclerosation) and, finally, the vessel diameter will be reduced (stenosation). According to the present state of scientific research, fat cells and inflammation cells will first become deposited on damaged vessel walls, and these cells, by inclusion of further fats, calcium salts and various cells, will develop into so-called plaques which cause a reduction of the vessel diameter. The decreased blood circulation will cause an undersupply to the organs and a resultant lack of oxygen.

Microangiopathy is caused by a change of the sorbite household in patients with diabetes. An increase of the sorbite concentration will entail a swelling of the inner skin of the vessel (intima), which in turn will lead to a narrowing of the vessel diameter and thus to a decreased blood circulation.

Arteriosclerosis as well as diabetic microangiopathy are the cause of microvascular diseases which should be diagnosed as early as possible. Hereunder, some explanations will be rendered on how a microvessel analysis, i.e. an analysis of the condition of the capillaries to the arterioles, is performed according to the present state of the art.

Microangiopathy can be detected in a non- or minimally invasive manner inter alia under the microscope or by mirror examination of the fundus of the eye. There also exist indirect indicators pointing to microangiopathic changes, such as e.g. low protein loss via the kidneys.

Mirror Examination of the Fundus of the Eye—Diabetic Retinopathy (Microvascular Angiopathy of the Retina):

When examining the fundus of the eye (opthalmoscopy), the ophthalmologist can observe the inner surface of the eyeball. With the aid of a magnifying glass, the ophthalmologist will inspect the interior of the eye via the pupil. The eye has to be illuminated by a light source. Basically, two techniques exist for examining the fundus of the eye:
Direct ophthahnoscopy: The ophthalmologist uses an electrical hand-held eye speculum (opthalmoscope). The light of this eye speculum is directed into the patient's eye in a manner allowing the ophthalmologist to visually observe the interior. The examination can be carried out relatively easily but, due to the strong magnification, will present only a small portion of the fundus of the eye. On the other hand, details in the center of the retina, such as the exit point of the optic nerve, the yellow spot (macula) and the central blood vessels, can be evaluated very precisely.
Indirect opthalmoscopy: The ophthalmologist holds a magnifying glass on his or her outstretched arm at a position in front of the patient's eye. The ophthalmologist will support the hand holding the magnifying glass on the patient's forehead and hold a light source in the other hand. By use of this technique, the image of the fundus of the eye will appear with a magnification of about 2.5 times and be turned upside down. The advantage over a direct examination resides in the larger survey and the improved depth of focus. However, the indirect technique requires somewhat more routine on the side of the opthalmologist. The opthalmoscope can also be installed in the central examination device of the ophthalmologist, the slit lamp (examination microscope). This makes it possible for the examining person to perform the examination using both eyes (binocularly) and will additionally improve the optical quality.

Nailbed Microscopy—Microangiopathy of the Acra, Largely Caused by Diabetes:

Particularly in the early stages of the disease, peripheral microangiopathy can be detected and evaluated in a very good manner by nailbed capillary microscopy or, better still, nailbed capillary microscopy of the video type. Capillary microscopy, as the only simple, non-damaging method, allows for direct examination and evaluation of the microcirculation of the skin and the visual appearance of the capillaries. When using video technology in the examination, it is further possible to detect dynamic processes in the capillaries. By capillary microscopy, the blood flow in the capillaries can be observed directly under the microscope. Capillary lesions can be represented by use of fluorescent colorants. This can be successfully performed on each site of the body surface, preferably on the groove of the nail bed. From the visual appearance and the spatial distribution of these smallest vessels and from the likewise observable blood flow, conclusions can be drawn on disturbances of the microcirculation.

Microalbumin in the Urine—Diabetic Nephropathy (Microangiopathy of the Kidney Vessels):

If the blood glucose has been on an increased level for a longer period, all proteins in the body are saccharified to an increased extent. The wall of the fine blood vessels in the kidneys also consists of proteins which form a very delicate net for filtration. If this wall has to take up the saccharified proteins, the wall will swell and the meshes will become coarser, thus reducing the filtering capacity of the kidney. Via the coarser meshes, larger molecules can escape into the urine. Albumin is one of the first proteins which in case of a functional restriction will pass via the kidneys into the urine. There, they can be detected. Presently, very sensitive measurement methods exist which are suited for detection of even very small quantities.

Biopsy (Minimal-Invasive):

In biopsy, a tissue sample is taken from the body. The pathologist will examine the sampled tissue under the microscope. Also chemical analyses, however, are counted among the examination methods. The findings obtained from biopsy allow for conclusions on the microscopic structure (the histology) of the tissue under examination.

Transcutaneous Measurement of the Oxygen Partial Pressure:

The transcutaneous measurement of the oxygen partial pressure is a non-invasive method for determining the oxygen partial pressure on the skin surface, allowing for a (rough) estimation of the blood flow situation. This examination method should always be performed in combination with another method so as to achieve a reliable diagnosis.

Hereunder, it will be briefly described how the macrovessel analysis, i.e. an analysis of the condition of the larger arterioles, is performed according to the present state of the art.

By use of vessel Doppler methods and other angiological-radiological as well as plethysmographic methods, it is possible to assess the condition of the larger vessels. These, however, are to be dealt with rather in the field of the diagnosis of macroangiopathies which, in turn, do not unambiguously indicate the existence of a microangiopathy.

Ultrasound:

Vessel diagnostics by means of ultrasound can be subdivided into acoustic and image-guided methods. The acoustic methods are usually understood in the sense of the so-called pocket Doppler methods. In such a method, the Doppler effect is used to trans-form the blood flow into an acoustic signal. For this purpose, one will detect the echo scattered by the erythrocytes—of an in-coupled sound wave, which echo has been shifted relative to the input signal by the Doppler frequency. Since changes in a vessel will result in changes of the blood flow, also the acoustic signal at the site above the change of the vessel will change. The image-guided Doppler method is based on the same regularities as the examination by pocket Doppler, with the difference that the registered frequency-shifted signal is transformed not into an acoustic signal but a visual signal.

With the aid of ultrasound diagnostics, however, stenosation and sclerosation can be generally detected only in the larger arteries (d>3 mm) Smaller vessels can hardly be represented through ultrasound, which is even less feasible for possible changes of such vessels. At the point, however, when a stenosation of the larger vessels has been detected, the irreversible stage of arteriosclerosis has already been reached.

Angiography:

Angiography is a method for vessel diagnostics which is based on the X-ray-diagnostic representation of the vessels. For this purpose, the patient will be injected an X-ray contrast medium which functions to highlight the blood flow in an X-ray picture. By use of this method, even small vessels (diameter up to about 1 mm) can be represented, thus allowing also for an early detection of arteriosclerotic changes. However, angiography is a complex method and is not without risks.

Coronary Angiography:

Coronary angiography is an angiography of the coronary vessels and thus a special type of X-ray examination wherein the coronary arteries are made visible. Via a cardiac catheter, the lumina of the coronary vessels are filled with an X-ray contrast medium which is injected into the coronary vessels. The contrast medium filling will be made visible by X-ray radiation and be documented on film material or, presently, mostly on digital storage media. Said filling serves for the diagnostics of the morphological conditions of the coronary vessels (coronary arteries) and for localizing of stenosis and of their type and extent.

Plethysmography:

Impedance plethysmography is a term for medical examination methods which are based on the measurement of the electrical AC resistance (impedance) of the tissue portion under examination. In this method, a current is fed into the patient's tissue via two electrodes. Due to the electric current, an electric field is built up in the tissue portion under examination, which field is varying under the influence of the arterial pulsation of the blood within the vessels. Impedance plethysmography is applied for determining the running time of the pulse waves and for analyzing the shape of the pulse wave because pathological changes in the arterial vessel system have an influence on the arterial pulse wave and the wave shape thereof. However, the method is suited only for the large vessels of the arms and legs but hardly for the smaller vessels (e.g. fingers and acra).

What is described as plethysmography is mostly the photometric measuring method wherein the light absorption of a tissue portion, which is dependent on the pulsation of the blood, will be recorded. The diagnostic possibilities correspond to those of impedance plethysmography, with the difference that this method can be used also acrally for the small vessels. For less pronounced stages of arteriosclerosis, however, the diagnostic recognition on the possibility of arteriosclerosis will not be significant and only in case of a distinct form, which is not reversible anymore, the diagnostic reliability is higher.

### SUMMARY

It is an object of the invention to provide a non-invasive method for reliable determination of microvascular diseases.

According to the invention, the above object is achieved by the features of claim 1.

In the method for non-invasive determination of microvascular diseases, a volume pulse development of a first blood constituent in a blood path will be determined. Further, a volume pulse development of a second blood constituent in a blood path, which second blood constituent differs from the first blood constituent, will be determined. Said second blood constituent can be e.g. the total hemoglobin in the blood. Subsequently, comparable reference points, such as e.g. local maxima, will be detected in the volume pulse development of the first blood constituent as well as in the volume pulse development of the second blood constituent. Then, there is detected a temporal difference for the detected difference points in the volume pulse developments of the first blood constituent and the second blood constituent.

It is a precondition for the method of the invention that the second blood constituent is bound to a erythrocyte, as is the case e.g. in a hemoglobin derivate. The first blood constituent must be significantly different from the second blood constituent with regard to its physical properties such as e.g. size, weight, density and so forth. Preferably, the first blood constituent is water. However, use can be made also of other blood constituents, such as e.g. lipids, free plasma proteins or the like, which are present in the plasma.

The method of the invention is based on the fact that microvascular diseases will lead, inter alia, to a change of the penneability of the blood vessels. Particularly, there will occur a decrease of the permeability to the erythrocytes in the capillaries. This is the case because the erythrocytes are larger than the actual diameter of the capillaries. Only by a high elasticity of the vessels but also of the erythrocytes, as well as by an unreduced cross section of the blood flow path, an unproblematic movement of the erythrocytes and thus a sufficient oxygen transport will be guaranteed. The gas exchange in the capillaries is effected by a very close contact and the largest possible surface abutment between the erythrocytes and the vessel wall. A change of the permeability to the erythrocytes in the capillaries will cause a temporal difference, e.g. between the hemoglobin volume pulse and the water volume pulse in the blood, which will propagate all the way into the arterioles because the much too small water molecules can move faster through a constriction site and, respectively, their velocity is independent from the elasticity of the vessel wall. From the detected temporal difference in the volume pulse developments of the first blood constituent and of the second blood constituent, e.g. of the hemoglobin, information with regard to the condition of the blood vessels can be derived. For instance, it is possible that a microvascular disease will be diagnosed if the amount of the temporal difference between the volume pulse of the first blood constituent and the volume pulse of the second blood constituent exceeds a specific threshold value. The setting of a suitable threshold value can be performed on the basis of diagnostic aspects.

Apart from a temporal difference between the water volume pulse and the volume pulse of the second blood constituent, there will frequently occur a change of the shape of the volume pulse developments if a microvascular disease exists. Thus, from the shape of the detected volume pulse development of the first blood constituent and of the volume pulse development of the second blood constituent, also information on the condition of the blood vessels can be derived.

Further information can be obtained by measuring the development of the flow velocities of the first blood constituent and of the second blood constituent. Measurement of the flow velocities is preferably performed according to the laser Doppler method.

The background of the method steps described so far resides in the fact that, due to microangiopathy, different blood constituents will pass the capillary system differently well and respectively with different velocities. Within a systolic cycle, this will cause short changes in concentration and velocity of the individual blood constituents relative to each other before the capillaries. These changes will continue backwards all the way to the arterioles. This has the consequence that the volume and/or flow pulses of the individual blood constituents, which preferably are to be registered simultaneously, will deviate from each other. The flow pulses are understood to be the flow velocities of the blood constituents. The deviations between the individual blood constituents can be temporal displacements but also particularly characteristic contour differences in the development of the flow velocity (flow pulse development) and/or in the volume pulse development. Volume pulses are preferably recorded plethysmographically, and flow pulses are preferably recorded by the laser Doppler method.

The flow of blood and blood constituents is generated primarily by contraction of the ventricles, i.e. by “squeezing”. Assuming that, ideally, there were no vessel resistance, only one flow pulse would exist. Due to the sudden increase of volume and due to the real vessel resistance, it is first, after the systole, the left-ventricular outflow tract (aorta) which will be widened. On the one hand, the aorta has the function of distributing the blood over the peripheral region of the body, and on the other hand, because of its elasticity, the aorta will change the pulsed blood flow at the entrance region of the aorta into a uniform flow. This effect is referred to as a Windkessel function. Thus, the ventricles as well as the Windkessel function are responsible for the blood flow and respectively the flow pulse. By the laser Doppler flow measurement, for instance, one can detect the flow pulse curve from which there can be derived the real flow velocity of the erythrocytes and other blood constituent in the vessels, i.e. how fast the particles are advancing.

Apart from the blood flow (flow pulse), also a pressure and respectively volume pulse is generated which can be registered as a pulse wave. Under the influence of the real vessel resistance, i.e. because, after the ventricle contraction, the blood cannot flow off in an unobstructed manner, there is caused a local widening of the vessels, propagating in the form of a pulse wave from the aorta to the periphery. Respectively associated therewith is a local increase of pressure and volume which can be measured by photo or impedance plethysmography. The pulse wave starts immediately after the ventricle contraction and is considerably faster than the blood flow (the average flow velocity is about 5-20 cm/s, the average pulse wave velocity is about 600-1000 cm/s).

Thus, the flow velocity and the pulse wave velocity are two different, mutually overlapping phenomena which can be detected and evaluated independently from each other. The flow velocity is mostly represented as a function of time v(t) but is occasionally also referred to as a flow pulse i(t).

The measurement of the volume pulse development of the first blood constituent and of the second blood constituent is preferably performed by the following method. First, at least two measurement radiations of two different wavelengths are emitted by a radiation source, wherein the emission of the measuring radiations can be performed continuously but preferably is performed sequentially. A fraction of the measuring radiation of each wavelength that is reflected and respectively transmitted by a body part to be examined, e.g. a human finger, is received by one or a plurality of light receivers, e.g. photodiodes. In the process, the light intensity is influenced by the wavelength-dependent absorption capacity of the water and the other blood constituent. The pulsatile development of the measuring radiations for both wavelengths is detected and stored. Preferably, an averaging is performed over a plurality of pulsation cycles.

The steps which are relevant for the invention, such as e.g. the determining of the volume pulse developments of the first blood constituent and of the further blood constituent from the intensity development for the two wavelengths used, and, further, the determining of comparable reference points in the volume pulse developments, and the determining of a temporal difference of the determined difference points, will be performed independently from the detection of the physical data on the patient's body. For instance, a measurement for detection of the absorption values at two wavelengths can be performed on the patient's body whereupon these absorption values will be stored for further processing. Now, with the aid of the above mentioned steps which are essential for the invention, the stored absorption values can be further processed at another site or at another time. The processing of the determined values can be performed, for instance, by a computation device such as e.g. a PC, which has no connection to the patient's body. Thus, the essential steps of the method of the invention can be carried out independently from the presence of the patient.

A suitable apparatus for performing the method of the invention is described in the patent application “Vorrichtung zum Ermitteln von Konzentrationen von Blutbestandteilen” filed by the Applicant. Also other suitable apparatus for the determining of volume pulse developments of different blood constituents can be used.

It is particularly preferred that said at least two used wavelengths comprise a first wavelength which is absorbed substantially only by the second blood constituent and not by the first blood constituent. This can be e.g. a wavelength in the range of about 500 to 600 nm. Radiation of this wavelength will be absorbed substantially only by the hemoglobin in the blood but not by the first blood constituent. Further, preferably, use is made of a second wavelength which will be absorbed substantially only by water but not by the second blood constituent. This can be e.g. a wavelength in the range of about 1100 to 1300 nm. This wavelength will be absorbed substantially only by the first blood constituent and not by the hemoglobin. By the use of such suitable wavelengths, it is rendered possible to determine a volume pulse development of the first blood constituent and a volume pulse development of the second blood constituent in a simple manner. The volume pulse development of the individual blood constituents herein corresponds to the measured intensity development of the emitted radiation for each wavelength.

Hereunder, a brief description will be given of the laser Doppler method for determining the flow velocity.

In flow measurement of the laser Doppler type, laser light will be irradiated into the tissue. At least a certain fraction of this light will be incident on the moving erythrocytes whereby the frequency of the light will be shifted due to the optical Doppler effect. The wave trains scattered back from the tissue will interfere on the photodetector of the sensor. On the basis of the resultant photo current, the Doppler frequency and thus a measure for the blood flow velocity can be determined.

Also on the basis of the development of the flow velocity which is measured by the laser Doppler method, a shape analysis of the determined curve can be performed in analogy to the shape analysis of the volume pulse curves.

With particular preference, examination of the shape of the measured volume pulse curves is performed with regard to amplitude development, maxima and minima, rises etc. Further, there can be performed an examination of the variability of the shape of the volume pulse curve between the individual periods of a wavelength. Further possible is an examination of the contour differences between the pulse curve for the first blood constituent and the pulse curve for the second blood constituent within a period. Based on the last-mentioned two steps, it is also possible to perform an examination of the total variability. In the examination of the temporal differences between the two volume pulse curves, it is also possible to use local minima apart from the maxima.

### BRIEF DESCRIPTION OF THE FIGURES

Preferred embodiments of the invention will be explained hereunder with reference to the Figures.

In the Figures, the following is shown:

FIG. 1 is a graphical representation of volume pulse developments of water and hemoglobin,

FIG. 2 is a schematic representation of a suitable apparatus for the determining of volume pulse developments of different blood constituents,

FIG. 3 is a representation of absorption coefficients of water and hemoglobin constituents,

FIG. 4 is a graphical representation of flow pulse curves in the arterial system, and

FIG. 5 is a graphical representation of parameters which can be determined on the basis of a pulse curve shape.

### DETAILED DESCRIPTION

A volume pulse development of the water fraction in a blood path is determined by emitting a measuring radiation of a wavelengths of about 1200 nm. For this radiation, there is detected the absorption of said wavelength by a body part to be examined, e.g. a human finger, over time. Since this wavelength is absorbed almost exclusively by the water fraction of the blood but hardly by the hemoglobin, the volume pulse development of the water fraction can be directly derived from the determined absorption development. In a corresponding manner, the volume pulse development for the total hemoglobin is determined at a wavelength of about 500-600 nm because, at this wavelength, there will occur only an absorption by the hemoglobin but only negligibly by the water in the blood.

To allow for a comparison of the two volume pulse developments to each other, it is preferred to determine comparable reference points, in this case local maxima, temporal differences as well as amplitude relationships between significant points of the respective intensity curve of the water fraction as well as in the intensity curve of the hemoglobin fraction. Subsequently, a temporal difference Δt of the detected local maxima in the intensity developments of the water fraction and of the hemoglobin fraction will be determined and, respectively, the ratio of the temporal differences as well as amplitude differences will be computed.

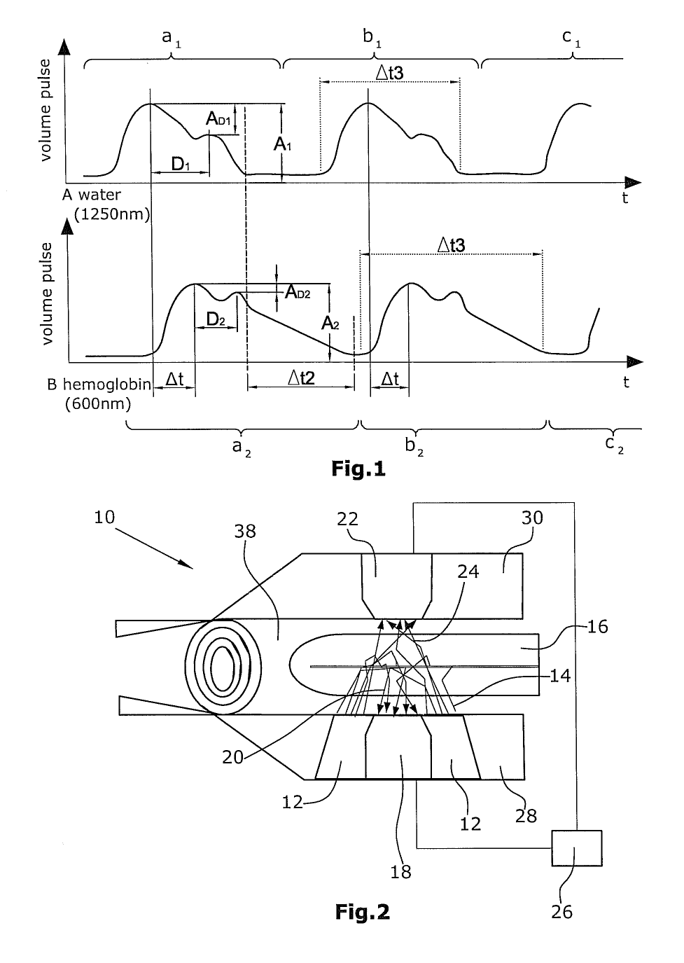

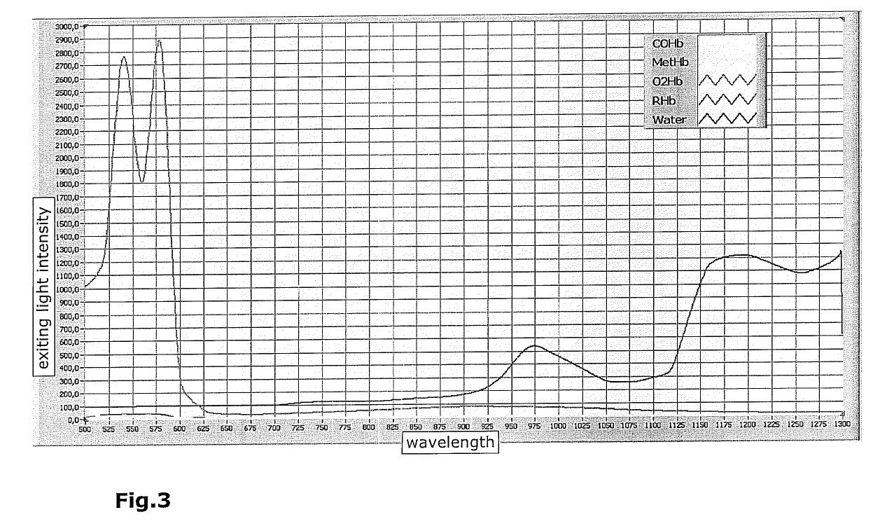

As evident from FIG. 1, the volume pulse development of the water will run ahead of the volume pulse development of the hemoglobin fraction by the value Δt. This means that the hemoglobin fraction in the blood will be delayed by specific changes of the blood vessels. From the extent of the delay (amount of Δt), one can derive information on the condition of the blood vessels. Particularly, it is possible in this manner to diagnose microvascular diseases in a non-invasive, quick and simple manner.

A further parameter for comparing the intensity curves for different wavelengths is e.g. the ratio of temporal differences within the respective intensity curve. Thus, the temporal distance D1 and respectively D2 of the two maxima—caused by the dikroty—of the intensity curves within a pulse period and the temporal ratio D1/D2 to be generated therefrom will be of relevance.

Further, the amplitude ratios can be compared to each other. For this purpose, it is suitable that the pulsatile amplitudes A1 and A2 for the two different wavelengths are adjusted to each other, i.e. are respectively set to 100%. Then, the relative amplitude differences A_{D1} and A_{D1} caused by the dikroty, relative to the 2nd main maximum of the pulse wave, can be set into a relationship to the maximum amplitude A1 and A2, and these ratios A_{D1}/A1 and A_{D1}/A2 can be compared to each other.

Further, the volume pulse developments of the individual blood constituents can be divided into individual periods (a₁, b₁, c₁, . . . ; a₂, b₂, c₂ . . . ), wherein the individual periods of the volume pulse development of a blood constituent (a₁, b₁, c₁, . . . ) are examined with regard to their variability. From the differences between the two periods of a volume pulse development, further information on the condition of the blood vessel can be derived. Further findings can be obtained from the differences between the shape of the volume pulse development of the water fraction (a₁) and the shape of the volume pulse development of the hemoglobin fraction (a₂) of the same period. Also a combination of the two last-mentioned comparative steps can be utilized for gaining more farreaching results.

For comparison of the different intensity curves for two wavelengths with regard to the temporal differences, the shape as well as the amplitude ratios, it will normally not be required to know the absolute intensity ratios because what counts will only be the relative changes of these parameters as compared to the maximum pulsatile intensity change.

In a further step, the flow velocity of the water and of the hemoglobin fraction can be determined e.g. by a laser Doppler method which likewise reacts in a sensitive manner to the different blood constituents, so that further information can be derived from the determined flow velocity.

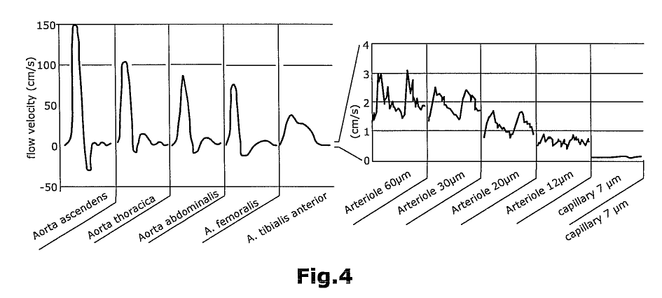

In FIG. 4, the flow pulses in the arterial system are represented, wherein the flow, which is still pulsating at the entrance to the arterial system, is taking on an ever more continuous character with increasing distance from the aortic valve (towards the right-hand side in FIG. 4). In the capillary vessels, the flow pulse is nearly fully damped.

FIG. 2 illustrates, by way of example, a suitable apparatus adapted to determine volume pulse developments of different blood constituents. The apparatus comprises a radiation source 12 as well as a first reflective radiation receiver 18 in the form of a photodiode, which are accommodated in a first accommodation element 28. Arranged opposite to said first accommodation element 28 is a second accommodation element 30 accommodating a second radiation receiver in the form of a photodiode 22. Between the first 28 and the second accommodation element 30, a receiving space 38 is formed which is suited to receive a body part 16 to be examined, e.g. a human finger. By means of said light source 12, a measuring radiation 14 of about 500-600 nm and 1100-1300 nm is radiated preferentially in a sequential manner. A fraction of said measuring radiation 14 will be reflected towards the reflective photodiode 18 while a further fraction 24 of the measuring radiation 14 will be transmitted towards the transmissive photodiode 22. It can be of advantage if the measuring radiation of the longer wavelength is captured by transmission, and the shorter wavelength by reflection.

The differences in the absorption of the various blood constituents for the wavelengths used, and thus of the received intensities, are based on the Lambert-Beer law. The calculation of the transmitted intensity is performed according to the formula

$$E = \frac{I}{{I}_{0}} = {10}^{- ɛ \cdot c \cdot d}$$

wherein
E=extinction
I=exiting/transmitted intensity
I₀=incident intensity
ε=molar extinction coefficient
c=concentration
d=layer thickness

The Lambert-Beer law describes how the radiation intensity, when passing through an absorbing substance, will behave in dependence on the concentration of the substance. In this regard, the extinction will result from the ratio between the transmitted light and the incident light fraction. In the present case, the diameter of the layer thickness d will undergo a slight change due to the blood pulsation and thus will cause a small intensity modulation which responds almost linearly to the change of the layer thickness. The linearity of the dependence is a direct consequence of a series development of the Lambert-Beer law, wherein already the linear member of the series development will describe, with sufficient precision, the interrelationship between the absorption and the layer thickness d. The change of the layer thickness in turn corresponds to the volume pulse development.

Besides, particularly in the evaluation of temporal differences of significant points of the recorded intensity developments, a linear interrelationship between the distance d and the intensity I is not at all required.

A suitable apparatus for performing the method of the invention is described especially in the patent application “Vorrichtung zum Ermitteln von Konzentrationen von Blutbestandteilen” filed by the Applicant. As far as further suitable apparatus exist for the determination of volume pulse developments of different blood constituents, these can be used as well. Thus, the invention also relates to an apparatus for determining the volume pulse developments of different blood constituents in order to detect microvascular diseases.

The determined absorption values for the two used waves of the measuring radiation 14 will be stored, followed by a renewed sequential emission of the two wavelengths of the measuring radiation 14. Upon each repetition of the emission of the measuring radiation 14, the detected absorption values will be stored for each wavelength, whereupon the stored absorption values will be combined into a representation of the temporal development of the absorption for each used wavelength of the measuring radiation 14. From these absorption developments, the volume pulse developments of the water as well as of the total hemoglobin can be determined.

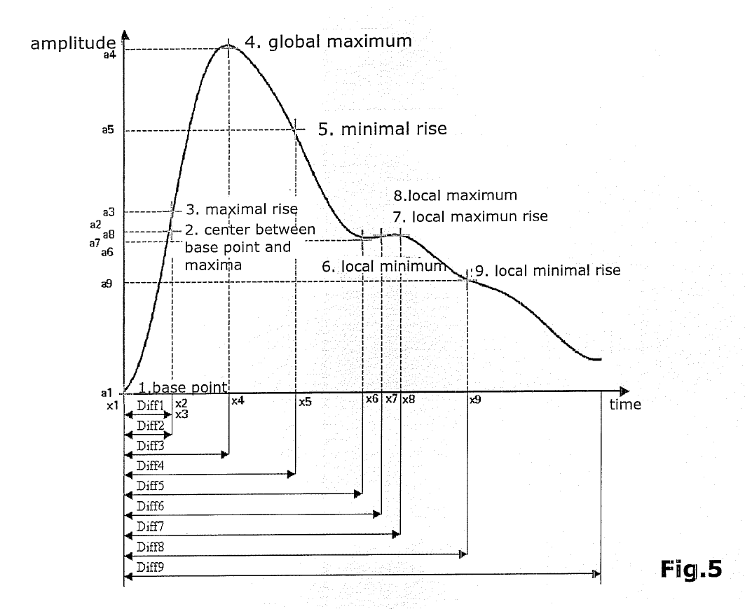

Examples of possible parameters which can be extracted from a pulse curve are shown in FIG. 5. These can be parameters from the volume pulse curve as well as from the development curve of the flow velocity. On the basis of these significant parameters, it is possible, as already described, to obtain information on the condition of the blood vessels.

What is claimed is:

1. A method for determining microvascular diseases, comprising:
determining a volume pulse development of a first blood constituent in a blood path,
determining a volume pulse development of a second blood constituent being different from the first blood constituent, in a blood path,
determining comparable reference points in the volume pulse development of the first blood constituent and in the volume pulse development of the second blood constituent, by means of a computation device,
determining a temporal difference (Δt) of said determined reference points in the volume pulse developments of the first blood constituent and the second blood constituent by means of a computation device, and/or determining amplitude and respectively temporal differences between the reference points in the volume pulse development of the first blood constituent and in the volume pulse development of the second blood constituent by means of a computation device, followed by the computation of the ratios of said determined amplitude and respectively temporal differences between the volume pulse developments of the first blood constituent and the second blood constituent by means of a computation device.

2. The method according to claim 1, wherein said determining of the volume pulse development of the first blood constituent and of the second blood constituent further comprising:
radiating at least two measuring radiations of two different wavelengths, by means of a radiation source;
receiving the measuring radiation of the different wavelengths that has been emitted from a body part to be examined, by means of one or a plurality of light receivers;
determining the pulsatile fraction of the received intensity caused by the blood pulsations and the resultant pulsatile absorption variations;
performing the last-mentioned method steps one or a plurality of times, the respective pulsatile intensity developments for each wavelength of the measuring radiation being stored for each repetition cycle;
combining the stored pulsatile intensity developments into a representation of the temporal development of the intensity for each used wavelength of the measuring radiation; and
determining the volume pulse developments of the first blood constituent and of the second blood constituent on the basis of the detected intensity developments of the different wavelengths of the measuring beams.

3. The method according to claim 2, wherein the measuring radiations are preferably radiated continuously and the reception of these measuring radiations is performed by at least two light receivers, a respective one of the used wavelengths being detectable by the light receivers either through the constructional design or by an attached filter.

4. The method according to claim 2, wherein the two measuring radiations are emitted in a clocked manner and the light receiver or the plurality of light receivers are activated in this clock regime so that a quasi-continuous intensity curve can be determined for each of the two wavelengths.

5. The method according to claim 2, wherein the light receiver or the plurality of light receivers are configured for detection either of the reflected or the transmitted measuring radiation or of both fractions of the measuring radiation emitted by the body part to be examined.

6. The method according to claim 2, wherein said at least two used wavelengths are a first wavelength which is absorbed substantially only by the second blood constituent and not by the first blood constituent, and a second wavelength which is absorbed substantially only by the first blood constituent and not by the second blood constituent.

7. The method according to claim 1, wherein said comparable reference points in the volume pulse developments of the first blood constituent and of the second blood constituent are local maxima or minima.

8. The method according to claim 1, wherein said second blood constituent is the total hemoglobin in the blood.

9. The method according to any claim 1, wherein said first blood constituent is the water fraction in the blood.

10. The method according to claim 1, wherein information on the condition of the blood vessels is derived from the amount of said temporal difference (Δt) or from the ratio of amplitude and temporal differences between the volume pulse developments of the first and second blood constituents.

11. The method according to claim 10, wherein a microvascular disease is detected if the amount of said temporal difference (Δt) exceeds a threshold value.

12. The method according to claim 1, further comprising:
measuring the flow velocity of the first blood constituent and of the second blood constituent.

13. The method according to claim 12, wherein information on the condition of the blood vessels is derived from said measured flow velocities of the first blood constituent and of the second blood constituent.

14. The method according to claim 1, wherein information on the condition of the blood vessels is derived from the shape of the determined volume pulse development of the first blood constituent and of the volume pulse development of the second blood constituent.

15. The method according to claim 1, wherein information on the condition of the blood vessels is derived from the shape of the development of the determined flow velocity of the first blood constituent and of the second blood constituent.

16. The method according to claim 12, wherein the determining of the flow velocity of the first blood constituent and of the second blood constituent is performed by the laser Doppler method.

17. The method according to claim 1, wherein the determining of the volume pulse development of the first blood constituent is performed preferably exclusively by transmission measurement and the determining of the volume pulse development of the second blood constituent is performed preferably exclusively by reflection measurement, the volume pulse development of the first blood constituent being determined by a radiation of a larger wavelength relative to the radiation for the second blood constituent.
